(19) United States
(12) Patent Application Publication
(10) Pub. No.: US 2001/0039015 A1
(43) Pub. Date: Nov. 8, 2001

(54) Nipple aspirate fluid specific microarrays

(76) Inventor: Edward Sauter, Philadelphia, PA (US)

Correspondence Address: CLIFFORD K. WEBER, ESQ, OFFICE OF UNIVERSITY COUNSEL, THOMAS JEFFERSON UNI, 1020 WALNUT STREET, SUITE 620, PHILADELPHIA, PA, 19107, US

(21) Appl. No.: 09/765,791

(22) Filed: Jan. 19, 2001

Related U.S. Application Data
(60) Provisional application No. 60/177,273, filed on Jan. 21, 2000.

(51) Int. Cl. C12M 3/00; C12M 1/34; C07H 21/04; C07H 21/02; C12Q 1/68

(52) U.S. Cl. 435/6; 536/23.1; 435/287.2; 435/288.3

(57) ABSTRACT

A method is provided for aid in the diagnosis of breast cancer. Nipple aspirate fluid (“NAF”) is collected from the breast via a modified breast pump device. Breast epithelial cells (“BECs”) and proteins present in NAF are isolated. Nucleic acid is extracted from the BECs. The nucleic acids and secreted proteins are analyzed via a low-density nucleic acid and peptidic biopolymeric array, respectively. The level of RNA transcription, protein expression, and heterozygosity is compared to that of normal control cells. This invention has particular application to the early detection of breast cancer and the study of individual responses to chemo- and radiotherapeutic exposures.

### CONTINUING APPLICATION DATA

This application claims priority under 35 U.S.C. § 120 based upon U.S. Provisional Application No. 60/177,273 filed on Jan. 21, 2000.

### FIELD OF THE INVENTION

The present invention generally relates to the fields of molecular biology and oncology and to a method of detecting breast cancer, and more particularly, to a method of detecting alterations in transcription in breast epithelial cells (“BECs”) shed into nipple aspirate fluid (“NAF”); to a method of detecting alterations in proteins present in NAF; to a method for detecting the loss of heterozygosity in BECs shed into NAF; and to a method of detecting the effects of therapeutic agents for the treatment of breast cancer.

### BACKGROUND OF THE INVENTION

Breast cancer is the most common noncutaneous cancer among women in the United States. Over forty-one thousand women in the U.S. die yearly from the disease. The only well-established procedures to screen subjects for breast cancer are physical examination and mammography. Unfortunately, physical examination does not identify a significant number of early breast cancers, and mammograms miss 10-40% of early breast cancers. (Giuliano, A. E., The Breast, In: Current Surgical Diagnosis & Treatment, 293-316, 1994 1994). Moreover, in the recently operated breast, mammography and breast examination are generally of little help in predicting residual disease.

A number of pathogenic factors in the diagnostic biopsy have been associated with residual disease/local failure in the breast, including positive margins, gross multicentricity, extensive intraductal carcinoma, an age under 35-40, and invasive lobular carcinoma. (Lagios, M. D., Semin Surg Onc, 8: 122-28, 1992; Harris, J. R. et al, Cancer of the Breast, In: Cancer: Principles and Practice of Oncology ₄^{th} ed., 1264-1332, 1993). Despite the currently available markers, approximately half of the women who undergo mastectomy for presumed residual disease will not have disease when the breast is investigated microscopically.

Although the early detection of breast cancer will lead to a higher cure rate, the ideal form of treatment is prevention. The prevention of breast cancer is hindered by the difficulty in identifying an effective agent. Effective agents are difficult to identify in part because of the long period required for breast cancer to develop and, consequently, the requirement for lengthy clinical trials to test the efficacy of the agent, if the end point is the prevention of cancer. One way to shorten the time required to find an effective agent is the identification of intermediate biomarkers, which are biological alterations in cells or tissues that occur between the time of initiation and tumor invasion. An agent that partially or completely reverses the intermediate biomarker back to a normal phenotype may be interrupting carcinogenesis. Evaluating the effect of the agent requires the analysis of tissue, cells, or non-cellular fluid.

Present efforts to evaluate the breast directly either through evaluation of tissue, individual cells, or extracellular fluid have been hindered because the analysis of these specimens generally requires an invasive procedure such as needle or excisional biopsy or mastectomy. A noninvasive method for evaluating the breast would be beneficial. The adult non-pregnant, non-lactating breast secretes fluid into the breast ductal system. (Keynes, G., Br J Surg, 11: 89-121, 1923) This fluid does not escape because the nipple ducts are occluded by smooth muscle contraction, dried secretions, and keratinized epithelium. Breast fluid can be obtained through aspiration of the nipple with a modified breast pump. (Petrakis N. L. et al, J Natl Cancer Inst, 54: 829-34, 1975) This fluid contains several types of cells, including exfoliated BECs. (King, E. B. et al, Am J Clin Pathol, 64: 728-38, 1975). Nipple aspiration, therefore, has the attractiveness of quickly, painlessly, and non-invasively obtaining both BECs (the cells at risk for transformation to breast cancer) and proteins secreted from the breast epithelium, which are highly concentrated in the fluid, in a manner that involves essentially no risk.

Cellular NAF samples can be obtained in the majority of subjects. (Sauter, E. R. et al, Br J Cancer, 76: 494-501, 1997). Abnormal NAF cytology as well as DNA index and cell cycle changes have been correlated with increased breast cancer risk (Sauter, E. R. et al, Br J Cancer, 76: 494-501, 1997; Sauter, E. R. et al, Br J Cancer, 81: 1222-27, 1999), while NAF with normal cytology is associated with a low risk for future breast cancer (Petrakis, N. L., Cancer Epidemiol Biomarkers Prev, 2: 3-10, 1993). Samples containing few or no BECs also are informative, for NAF cytology of low cellularity has been associated with low breast cancer risk. (Sauter, E. R. et al, Br J Cancer, 76: 494-501, 1997; Petrakis, N. L., Cancer Epidemiol Biomarkers Prev, 2: 3-10, 1993). Malignant NAF cytology obtained from mastectomy specimens aspirated immediately after surgical removal was 100% specific for the presence of residual cancer in the breast. (Sauter, E. R. et al, Br J Cancer, 81: 1222-27, 1999). Secreted proteins in NAF, such as prostate-specific antigen (“PSA”), (Sauter, E. R. et al, Cancer Epidemiol Biomarkers Prev, 5: 967-70, 1996), and insulin-like growth factor binding protein type-3 (“IGFBP-3) (Sauter, E. R. et al, Proc Am Assoc Cancer Res, 39: 1590A, 1998) can be analyzed and have been shown to be associated with breast cancer risk.

The loss of heterozygosity (“LOH”), which is the loss of one or more alleles on a chromosome, is commonly found in tumor tissue when compared to unaffected tissue containing heterozygous alleles. Epidemiologic studies have identified a variety of candidate precursor breast lesions, including usual ductal hyperplasia (“UDH”), atypical ductal hyperplasia (“ADH”), and ductal and lobular carcinoma in situ (“DCIS”, “LCIS”), although it is not clear if these lesions, with the exception of DCIS, are true precursors of or rather merely indicators of increased breast cancer risk.

Recent studies suggest that LOH can be found in in situ carcinoma, ADH, and UDH of the breast (O'Connell, P., et al, J Natl Cancer Inst, 90:697-703, 1998), with the frequency increasing with disease progression toward invasive cancer. Using PCR amplification of candidate microsatellite regions, LOH has been found in at least one locus in 37% of UDH and 42% of ADH lesions in breasts free of invasive cancer. In DCIS without invasive cancer, LOH was found in 70% of noncomedo and 79% of comedo lesions. Among specimens containing invasive cancer, 37% of UDH, 45% of ADH, 77% of noncomedo DCIS and 80% of comedo DCIS lesions shared LOH with synchronous cancers at ≧ one locus. A recent report evaluating allelic loss at chromosome 11q13 in lobular lesions identified LOH in 10% of isolated atypical lobular hyperplasia (“ALH”), 11% of specimens with ALH and LCIS, and 50% of specimens containing LCIS and invasive lobular carcinoma (“ILC”). LOH in premalignant lobular lesions, therefore, may indicate a predisposition of the lesion to progress to invasive cancer.

Identifying the alterations in gene expression that are associated with breast cancer, including those involved in tumor development and progression, is a prerequisite, not only for a full understanding of breast cancer, but also for the development of new rational therapies. Genes characteristic of cancerous cells are often host genes that are abnormally expressed. Thus, it is often the case that a particular protein marker for a given cancer is expressed at high levels in the cancerous tissue while being expressed elsewhere in the body at low levels. Such changes in gene expression often predate the presence of gross changes observable by cytology. Traditionally, tumor classification has been based on the microscopic appearance or morphology of the tumor. This diagnostic classification scheme has limited capacity to predict treatment responsiveness and to offer prognosis for long-term survival from malignancies with similar histopathological characteristics. “Biochips” or arrays of binding agents, such as polynucleotides and peptides, have become an important tool in the biotechnology industry and related fields. These arrays, in which a plurality of binding agents are deposited onto a solid support surface in the form of an array or pattern, find use in a variety of applications, including drug screening, nucleic acid sequencing, and mutation analysis. One important use of biochips is in the analysis of differential gene expression. In such assays, “targets” from analogous cells, tissues or organs of a healthy and diseased organism are hybridized to nucleic acid or peptidic probe fragments that are immobilized on a substrate. Differences in the resultant hybridization patterns are related to differences in gene expression (RNA or protein) or gene sequence or copy number (DNA) in the two sources. Monitoring these patterns using such microarrayed grids of nucleic acid or peptide fragments is a powerful tool for simultaneously analyzing the expression, sequence, or copy number of many genes or gene fragments. In the oncology context, such assays ultimately allow for tumor classification (phenotyping) based upon molecular markers (DNA, RNA, or protein) and individualized screening of chemotherapeutic, chemopreventitive, and radiotherapeutic agents. (Sgroi, D. C. et al, J Natl Cancer Inst, 67: 277-84, 1999). As an adjunct tool to existing diagnostic methodologies, microarray studies will increase the sensitivity of NAF cytology in determining the benign or malignant nature of a breast lesion.

The present invention utilizes such a biochip system to assess, diagnose, and treat cancer, particularly breast cancer. In the present invention, the biochip is used to analyze samples of NAF, thereby taking advantage of a noninvasive approach to the early detection and diagnosis of breast cancer and the assessment of individual responses to chemo- and radiotherapeutic exposures.

### DEFINITIONS

The term “apoptosis related genes” as used herein includes bcl-2, bax, TRPM-2, TIPM-1, TIPM-2, ICE genes, c-myc, p53, K-ras, COX-1, COX-2, MDM-2, p21, p27, p16/INK4, PCNA, H-ras, MMP-1, ERK-1, ERK-2, and NF_{k-B}.

The term “insulin-like growth factor/human kallikrein system genes” as used herein includes IGF-1; IGFBP-1, -2, -2, -4, -5, -6, -7, -8; IGFR-1, -2; IGF II, PSA; hK2, hK6, and hK10.

The term “other key genes of interest” includes estradiol; estrone; 17α-OH progesterone; MMP2; MMP9; erbB-1; HER-2/new; hTERT; erbB-2; VEGF; PGE₂; hst-1; KDR; int-2; cyclin D1, 2, 3; CEA; H-ras; K-ras; and mammoglobin.

The term “key NAF proteins” includes PSA; IGFBP-1, -2, -4, -5, -6; hK2; IGFBP-3; and p53.

### SUMMARY OF THE INVENTION

Low-density, biopolymeric microarrays and kits including the same as well as a method for their use in assays involving NAF are provided. The subject microarrays have a plurality of biopolymeric probes, e.g., nucleic acids or peptides and are preferrably cDNA, peptides, or DNA microsatellites that correspond to genes or fragments of genes with proven importance in breast cancer or NAF proteins of interest. Breast cancer genes of interest that may be represented on the array include: oncogenes, apoptosis genes, genes associated with early response, and genes involved in development and differentiation of breast ductules/alveoli. Proteins of interest that may be represented on the array include those implicated in the insulin-like growth factor and kallikrein systems. The subject microarrays can be used to monitor the expression level of large numbers of genes and proteins simultaneously and to identify genetic variants, mutations, and polymorphisms. This information may be used to determine gene function, understand the genetic basis of breast cancer, diagnose breast cancer, and develop and monitor the activities of therapeutic agents.

### BRIEF DESCRIPTION OF THE FIGURES

FIG. 1. Diagram depicting an iteration of a low-density NAF microarray.

FIG. 2. Photomicrographs of cytological preparations of NAD (Papanicolaou stain, ×1000). A: Foam cells, a frequent constituent of NAF, which may represent apoptotic epithelial cell; B: cluster of normal epithelial cells (hyperplasia without atypia); C: atypical hyperplasia; D: malignant epithelial cells.

FIG. 3. Photomicrographs illustrating that the capture technique of laser microdissection is able to selectively (outlined area) collect BECs without disrupting surrounding foam cells.

FIG. 4. Autoradiograph showing RNA isolation from NAF. mRNA was isolated from subject RM and reverse transcribed to cDNA using RT-PCR. Total RNA was isolated from subject CA2. The quality and purity of the RNA obtained were evaluated using a 3′-oligo-dT uniprimer (3′) and multiplex RT-PCR (MPCR). RNA isolation was compared to total RNA from human placenta (HP).

FIG. 5. Autoradiographs illustrating low density microarrays using NAF.

FIG. 6. Identification of breast cancer biomarkers by analysis of NAF proteins bound to chemically defined surfaces.

### DETAILED DESCRIPTION OF THE INVENTION

Biopolymeric microarrays relating to breast cancer as well as a method for their preparation and use is provided. In the subject microarrays, a plurality of biopolymeric probes are stably associated with the surface of a solid support. The biopolymeric probes are either nucleic acids or peptides. In the preferred embodiments, the polymeric probes are cDNA, chemicals, microsatellites of DNA, or peptides that correspond to breast cancer genes or gene fragments of interest or proteins that are present in NAF. The subject microarrays find use in a variety of applications and are particularly suited for use in high throughput gene and protein expression analysis applications, particularly population studies. In further describing the subject invention, the subject microarrays will be described first, followed by a description of how the subject microarrays can be prepared, and a discussion of their use in a representative binding assay.

Array Structure

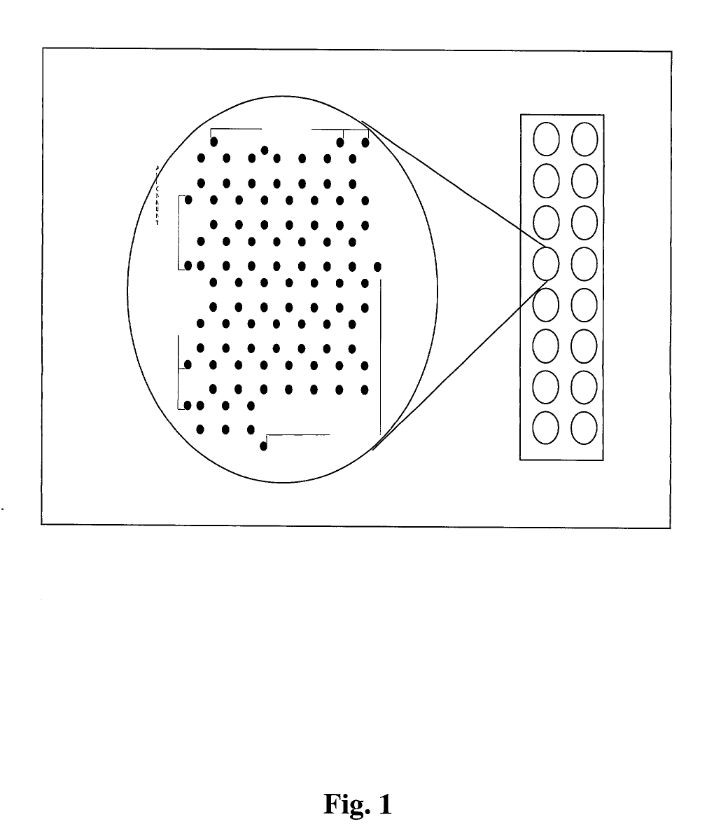

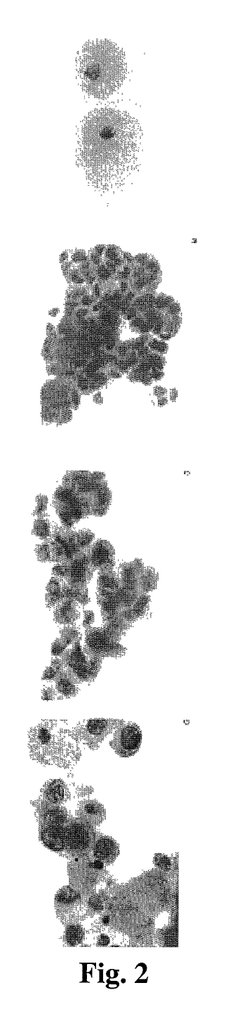

The microarrays of the subject invention have a plurality of biopolymeric probes stably associated with a surface of a solid support. The biopolymeric probes of the subject microarrays are nucleic acids, such as deoxyribonucleic acids, ribonucleic acids, peptide nucleic acids and the like, or peptides, such as antibodies (e.g., polyclonal, monoclonal and binding fragments thereof), peptides with high affinity to the targets, as well as analogues and mimetics thereof, ligands, receptors, and the like. The nucleic and peptide probes may be obtained from a natural source or synthesized using available technologies. The probe spots on the microarray may be any convenient shape, but will typically be circular, elliptoid, oval, annular, or some other analogously curved shape where the shape may be a result of the particular method employed to produce the microarray. The density of the probe positions, including calibration and control probes, on the surface of the support is selected to provide for adequate resolution of binding events, where the density will generally range from about 8-200 probes/microarray and but does not exceed 3000 probes/microarray. The probe positions may be arranged in any convenient pattern across or over the surface of the microarray, such as in rows and columns so as to form a grid, in a circular pattern, and the like, where generally the pattern of positions will be present in the form of a grid across the surface of the solid support. In the microarrays of the subject invention, a single pattern of spots may be present on the microarray or the microarray may comprise a plurality of different probe position patterns, each pattern being as defined above. When a plurality of probe position patterns are employed, the patterns may be identical to each other or different. (FIG. 1)

In the subject microarrays, the probes are immobilized on the surface of a solid support. By immobilized it is meant that the probes maintain their position relative to the surface of the solid support under hybridization and washing conditions. As such, the probes can be non-covalently or covalently stably associated with the support surface. The solid substrate of the subject microarrays may be fabricated from a variety of materials. The materials from which the substrate is fabricated ideally should exhibit a low level of non-specific binding of target during hybridization or specific binding events. Specific materials of interest include: glass, plastics, metals, nylon, nitrocellulose, polypropylene, etc. The configuration of the substrate of the subject microarrays may take a variety of configurations depending on the intended use of the microarray. Generally, an overall rectangular configuration is preferred.

The solid substrate of the subject microarrays comprises at least one surface on which a pattern of probe molecules is present, where the surface may be smooth or substantially planar, or have irregularities, such as depressions or elevations. The surface on which the pattern of probes is presented may be modified with one or more different layers of compounds that serve to modulate the properties of the surface in a desirable manner. The microarrays of the subject invention may be incorporated into a structure that provides for ease of analysis, high throughput, or other advantages, such as in a biochip format, a multiwell format, etc.

Biopolymeric Markers

A critical feature of the subject microarrays is that all of the probes of the microarray correspond to NAF BEC genes or fragments of genes that are of interest, particularly genes that have been identified as having an important role in breast cancer, or proteins secreted into NAF. Candidate molecular markers for breast tumor phenotyping include those genes involved in apoptosis, development and differentiation of breast ductules/alveoli, and the early response pathway. Specific breast genes that may be represented on the microarrays of the subject invention include those listed in Table 1. In many preferred embodiments, the microarrays of the subject invention will include genes associated with the induction or inhibition of apoptosis: bcl-2, bax, TRPM-2, TIPM-1, TIPM-2, ICE genes, c-myc, p53, K-ras, COX-1, COX-2, MDM-2, p21, p27, p16/INK4, PCNA, H-ras, MMP-1, ERK-1, ERK-2, and NF_{k-B}. In another preferred embodiment, the subject microarray will include genes involved in the insulin-like growth factor system/human kallikrein system: IGF-1; IGFBP-1, -2, -2, -4, -5, -6, -7, -8; IGFR-1, -2; IGF II, PSA; hK2, hK6, and hK10. In another preferred embodiment, all of the genes listed in Table 1 are present on the microarray. In a further embodiment, the microarray of the subject invention will include peptides that specifically bind to the proteins found in NAF listed in Table 2.

TABLE 1
KEY NAF GENES
Apoptosis-Related Genes

| | | | | | |
|---|---|---|---|---|---|
| Bcl-2 | TRPM-2 | TIPM-1, -2 | COX-1, -2 | p27 | ERK-1, -2 |
| ICE genes | bax | K-ras | MDM-2 | p16/INK4 | NF_{k-B} |
| c-myc | p53 | H-ras | p21 | PCNA | |

Growth Factor System Genes

| | |
|---|---|
| IGF-1 | hK2 |
| IGFBP-1, -2, -3, -4, -5, -6, -7, -8 | hK6 |
| IGFR-1, -2 | hK10 |
| IGF II | PSA |

Other Key Genes of Interest

| | | |
|---|---|---|
| Estradiol | Estrone | 17α-OH progesterone |
| MMP2 | MMP9 | erbB-1 |
| HER-2/new | hTERT | erbB-2 |
| VEGF | PGE₂ | hst-1 |
| KDR | int-2 | cyclin D1, 2, 3 |
| CEA | H-, K-ras | mammoglobin |

TABLE 2
KEY NAF PROTEINS

| | |
|---|---|
| PSA | IGFBP-1, -2, -4, -5, -6 |
| hK2 | IGFBP-3 |
| p53 | |

The subject microarrays typically comprise one or more additional probes that are not breast cancer genes or NAF proteins of interest. Other probes that might be present on the substrate surface include internal housekeeping genes, external unrelated genes, and the like. These probes serve as control genes, the function of which is to ensure the quality of the data. Specific housekeeping genes of interest include: β-actin and GAPDH. Specific external, unrelated genes of interest include: yeast genome, arabidopsis, and human 18s rRNA sequences.

Array Preparation

Microarrays may be prepared using methods known in the art. The solid substrate or support can be fabricated according to known procedures, where the particular means of fabrication depends on the material from which the support is made. The pattern of probe molecules is then prepared and stably associated with the surface of the support. The probe molecules may be isolated from cells, tissues, or organisms using standard techniques. Such methods typically involve tissue/cell homogenization, nucleic acid/protein extraction, chromatography, centrifugation, affinity binding, etc. The probe molecules may be further treated in order to improve hybridization and detection or enhance association with the surface of the support. Such treatments might include: reverse transcription, nuclease treatment, DNA amplification, etc.

Following stable placement of the pattern of probe molecules on the support surface, the resultant microarray may be used as is or incorporated into a biochip, multiwell or other device for use in a variety of binding applications.

Sample Analysis

Expression analysis applications using the subject microarrays generally involve the following steps: (1) preparation of a target, (2) contact of the target with the array under conditions sufficient for the target to bind with the corresponding probe, (3) removal of any unbound target, and (4) imaging.

Target preparation depends on the specific nature of the target, e.g., whether the target is nucleic or peptidic. A variety of different protocols may be used to generate labeled biopolymeric targets for detection in the imaging step. Labels may be either directly detectable, such as isotopic and fluorescent moieties incorporated into a moiety of the target, or detectable through combined action, such as labels that provide for a signal only when the target with which they are associated is specifically bound to a probe molecule. In one of the preferred embodiments, total RNA is isolated and amplified, where necessary, and labeled with radioactive probe.

Following its preparation, the labeled target solution is contacted with the microarray under conditions sufficient for binding between the targets in the sample and the probes on the microarray, where such conditions can be adjusted, as desired, to provide for an optimum level of specificity in view of the particular assay being performed. The method of achieving contact depends on the configuration of the array. Generally, the target sample will be a fluid sample and contact will be achieved by introduction of an appropriate volume of the fluid sample onto the microarray surface, where introduction is via an inlet port, deposition, dipping the microarray into a fluid sample, etc. Contact of the target solution and the microarray must be maintained for a period of time sufficient for binding between the targets and the probes to occur. Such time will vary depending on the nature of the targets and the probes. In one of the preferred embodiments, the labeled targets are incubated with the microarray so that the target sequences hybridize to complementary polynucleotide probes of the microarray. Incubation conditions can be adjusted so that hybridization occurs with precise complementary matches or with various degrees of less complementarity.

Following binding of probe and target, the nonhybridized labeled targets are removed from the support surface by washing. The resultant hybridization patterns of labeled target on the surface of the microarray may be visualized or detected in a variety of ways, with the particular manner of detection being dependent upon the particular label of the target. Representation detection methods include scintillation counting, fluorescence measurement, calorimetric measurement, radioactive probe quantification, etc. Hybridization patterns can be compared to identify differences between patterns. Where microarrays in which each of the different probes correspond to a known gene, any discrepancies can be related to a differential expression of a particular gene in the sources being compared. In one of the preferred embodiments, after removal of the nonhybridized probes, a scanner is used to determine the levels and patterns of signal. The scanned images are examined to determine the degree of complementarity and the relative abundance of each polynucleotide sequence on the microarray.

As such, the subject microarrays can find use in a variety of applications, including profiling differential gene expression in NAF and discovering potential therapeutic and diagnostic drug targets. The findings can be used both to screen for new or recurrent disease or to evaluate response to therapy, as with a chemotherapeutic or chemopreventive agent.

Kits

Also provided for are kits for performing binding assays using the subject microarrays, where kits for carrying out differential gene expression analysis assays are preferred. Such kits according to the subject invention will at least comprise a microarray according to the subject invention, where the microarray may simply comprise a pattern of target molecules on a planar support or be incorporated into a multiwell configuration, biochip configuration, or other configuration. The kits may further comprise one or more additional reagents for use in the assay to be performed with the microarray, where such reagents include: probe generation reagents, reagents used in the binding step, signal producing system members, etc. The kits may further comprise a modified breast pump for collection of NAF.

The following examples are offered by way of illustration and not by way of limitation.

### EXAMPLE 1

Measurement of Gene Expression Methods

NAF Specimen Collection

NAF is obtained using a modified breast pump. The device consists of a 10-ml syringe attached to the end of a no. 4 endotracheal tube over which is placed a respiratory humidification adapter. Subjects are seated in a comfortable position, and the breast nipple is cleansed with alcohol. After the alcohol evaporates, a warm, moist cloth is placed on each breast. After 1-2 minutes, the cloths are removed, the patient compresses her breast with both hands, and the plunger of the syringe is withdrawn to the 7-ml level and held for 15 seconds or until the patient experiences discomfort. Fluid in the form of droplets is collected in capillary tubes, with samples from each breast collected separately. The quantity of fluid varies from 1 to 200 μl.

Occasionally, keratin plugs rather than NAF are obtained after suction is completed. The plugs are removed with an alcohol swab and suctioning repeated. At times, suctioning has to be performed two or three times to remove all of the plugs. To obtain additional fluid, the nipple is gently compressed between two fingers. One or two additional droplets of fluid often appears.

For subjects with invasive cancer, the mastectomy specimen can be aspirated immediately after removal from the chest wall. Aspiration of mastectomy specimens is performed in a fashion similar to aspiration of the intact breast, with the exception that warm cloths are not used on the mastectomy specimens.

NAF Specimen Preparation

Half of the specimens collected in capillary tubes are placed in Trizol solution and then in −80° C. for future RNA microarray analysis. The remaining specimens collected in capillary tubes are rinsed into containers holding 3% polyethylene glycol in denatured alcohol (Shandon Lipshaw, Pittsburgh, Pa.). The fixed specimens are then cytocentrifuged onto 10 glass slides. Three of these slides are used for cytologic examination, while the remaining seven slides are stored for microarray analysis. The slides selected for cytologic examination are washed twice in 95% ethanol for 5 minutes each, rehydrated in tap water, and stained by the Papanicolaou (Pap) method. (FIG. 2).

The Pap-stained slides are examined to determine whether the NAF sample is cellular, defined as containing at least 10 BECs on a slide, and/or homogeneous, i.e., containing only BECs. Approximately 60-70% of NAF specimens are cellular. If cytologic examination reveals the NAF sample to be homogeneous, further analysis or RNAis performed using the NAF specimens stored in Trizol. DNA analysis is performed using cells on slides, and protein analysis using NAF stored in capillary tubes.

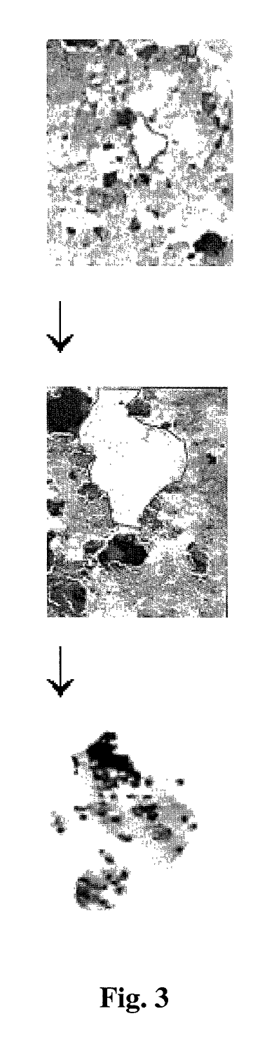

Laser Capture Microdissection (“LCM”) for RNA and DNA Analysis of Heterogeneous Populations of Cells

Where cytologic examination reveals the NAF specimen to be heterogeneous, however, the NAF cytospin specimens are laser dissected with either a PixCell I or II LCM system (Arcturus Engineering, Mountain View, Calif.) for collection of a pure population of BECs, thereby eliminating heterogeneity concerns. (FIG. 3) Following the standard protocol of Emmert-Buck, M. R. et al (Science, 274: 998-1001, 1996), modified as necessary for cytospin specimens, approximately 100-10,000 BECs generally can be “laser captured” from the cellular NAF specimens without disruption of the surrounding foam cells.

Biomarker Analysis

The RNA microarray technique has been described by Sgroi et al (J Natl Cancer Inst, 67: 277-84, 1999). A brief overview is provided.

RNA Extraction

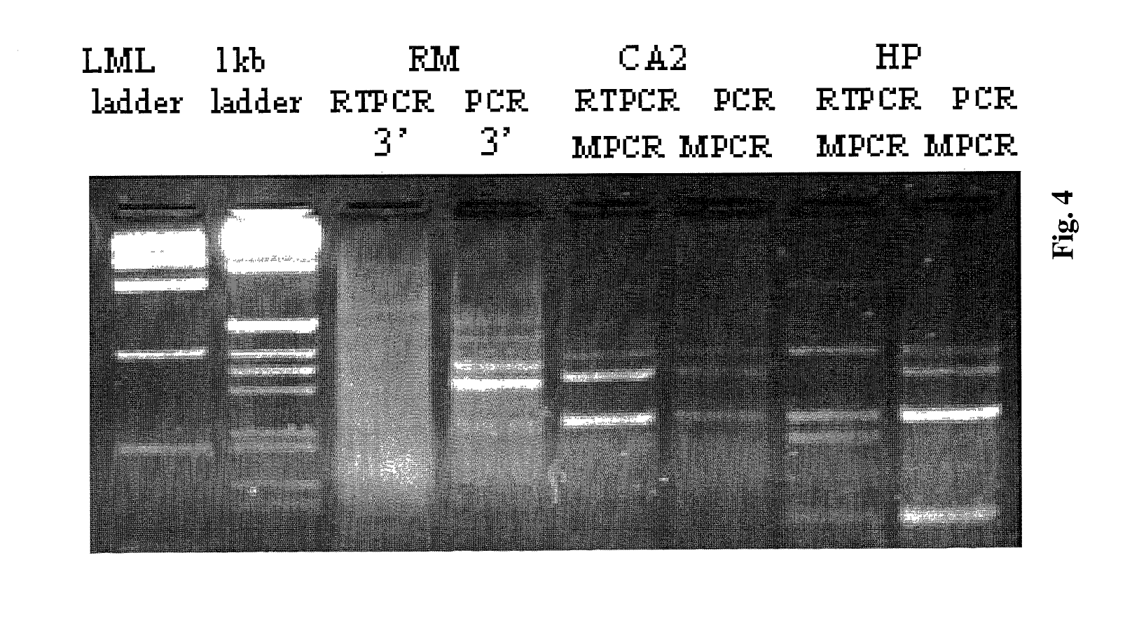

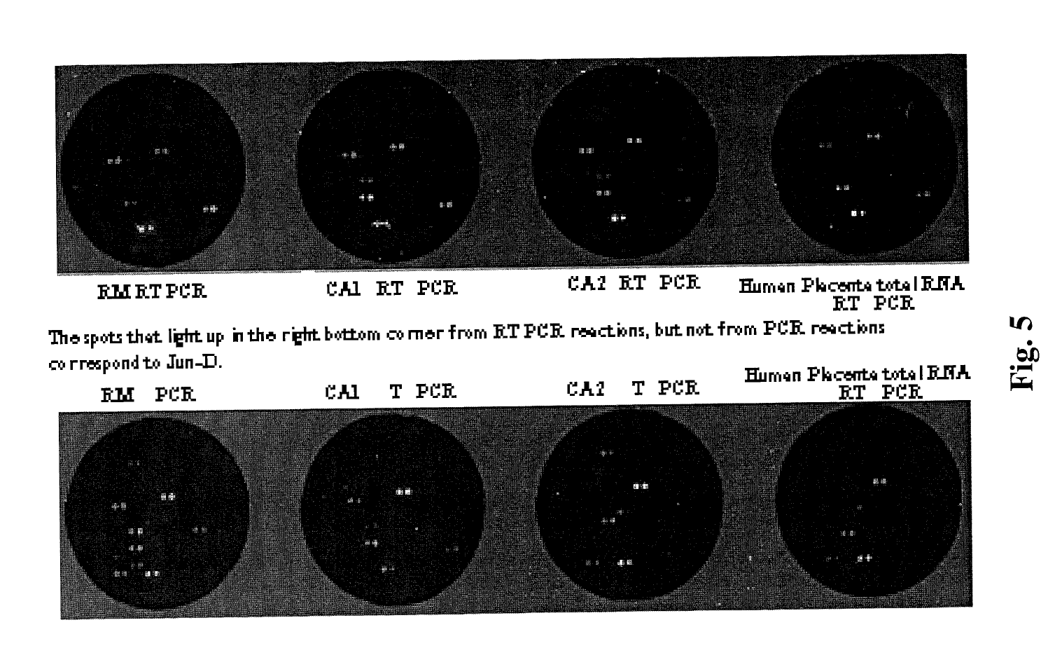

The total RNA from each NAF sample (FIG. 4) is obtained by means of a modification of the RNA microisolation protocol previously described by Emmert-Buck, M. R. et al (Science, 274: 998-1001, 1). For LCM specimens, the transfer film and adherent cells are incubated in guanidinium isothiocyanate buffer at room temperature, while from the NAF cells that were not laser captured are incubated with guanidinium isothiocyanate buffer. After incubation, the RNA is extracted with phenol/chloroform/isoamyl alcohol and precipitated with sodium acetate and glycogen carrier (10 μg/l) in isopropanol. After initial recovery and resuspension of the RNA pellet, a deoxyribonuclease (DNAase) step is performed for 2 hours at 37° C. using 10 units of DNase (GenHunter, Nashville, Tenn.) in the presence of 10 units of RNase inhibitor (Life Technologies, Gaithersburg, Md.), followed by re-extraction and precipitation. The pellet is resuspended in 27 μl RNase-free H₂O.

aRNA Amplification (if Necessary) and Labeling

In specimens having insufficient cellularity to directly perform microarray analysis, aRNA amplification is performed. The aRNA amplification method is a linear rather than an exponential amplification (in contrast to RT-PCR, for example), and as a result better retains the original MRNA abundance information through the amplification process. A single round of aRNA amplification is unlikely to significantly bias the relative mRNA species abundance in a given sample, and is a convenient means to permit microarray analyses of samples of approximately 1-50 ng starting RNA. One round of amplification is thought to amplify the RNA approximately 2000-fold; a second round, one million fold. Where necessary, aRNA amplification of the RNA is performed using the protocol previously described by Brooks-Kayal, A. R. et al (Nat Med, 4: 1166-72, 1998 [published erratum appears in Nat Med, 5: 590, 1999]).

Total RNA is added to a solution containing T7-oligo-d(T), incubated for 1 hour, and phenol/chloroform/100% EtOH added. The pellet is heated to 95° to separate RNA/DNA, cooled, a solution including DTT, T4 DNA polymerase and Klenow is added and incubated overnight. To remove ssDNA, S1 buffer and S1 nuclease is added, the solution incubated, and the specimen phenol/chloroform/EtOH extracted. To blunt end the cDNA, a solution including dNTPs, DTT, T4 polymerase, and Klenow is added, the solution incubated, and phenol/chloroform/EtOH extracted. For conversion to RNA from ds-cDNA, a solution including DTT, dNTPs, α-[³³P]-CTP, T7 RNA polymerase is added, and the solution is incubated at 37° C. Phenol/chloroform/EtOH is extracted and the pellet resuspended in DEPC H₂O and stored at −80° C. A second round of amplification ias performed if necessary.

Hybridization

The labeled RNA is denatured and hybridized to the cDNA gene filter as follows: The filters are pre-hybridized at 42° C. in a roller oven (Hybaid, Midwest Scientific, St. Louis, Mo.) with 1.0 μg/ml poly-dA (Research Genetics) and 1.0 μg/ml Cot1 DNA (BRL/Life Technology) in 5 ml in Microhyb solution (Research Genetics) for at least 2 hours. After overnight hybridization with the radiolabeled probe, the filters are washed twice at 50° C. in 2×SSC, 1% SDS for 20 minutes and once at room temperature in 0.5×SSC, 1% SDS for 15 minutes. The filters are then exposed overnight to a Packard screen and scanned at 50-micron resolution in a Phosphor-imager instrument (Cyclone Instrument from Packard, Meriden, Conn.). (FIG. 5) Each hybridization is performed in duplicate. After each hybridization, the filters are stripped by boiling in 0.5% SDS solution and scanned for residual leftover hybridization.

Printing of Low-Density Microarrays

Microarrays are printed using a Hamilton 2000 with a 4 pin (or similar) printer, narrow gauge print head and home-written C++ macros for 16 well slide or 96 well plate printing: 150 μM, 300 μM on center. Duplicate copies of probes along with a series of house keeping genes, including β-actin, GAPDH and alignment markers are printed onto epoxysilane surfaces. The microarrays are stable at 4° C. until their use.

### EXAMPLE 2

Protein Marker Identification Method

Analysis of protein biomarkers using a protein chip has been described by Wright et al (2000). A brief overview is provided.

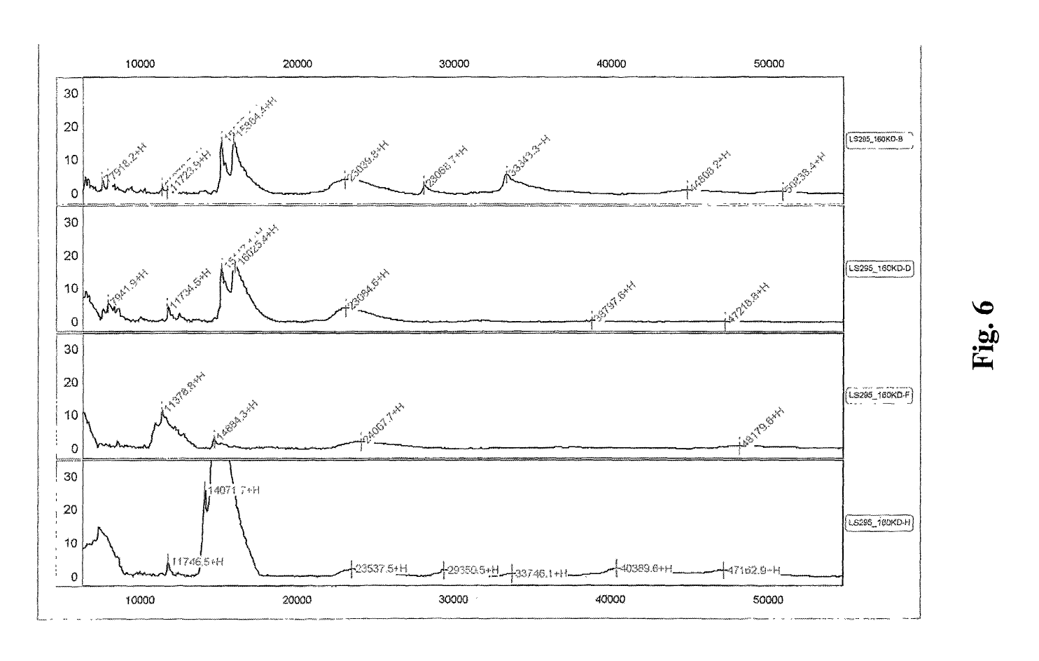

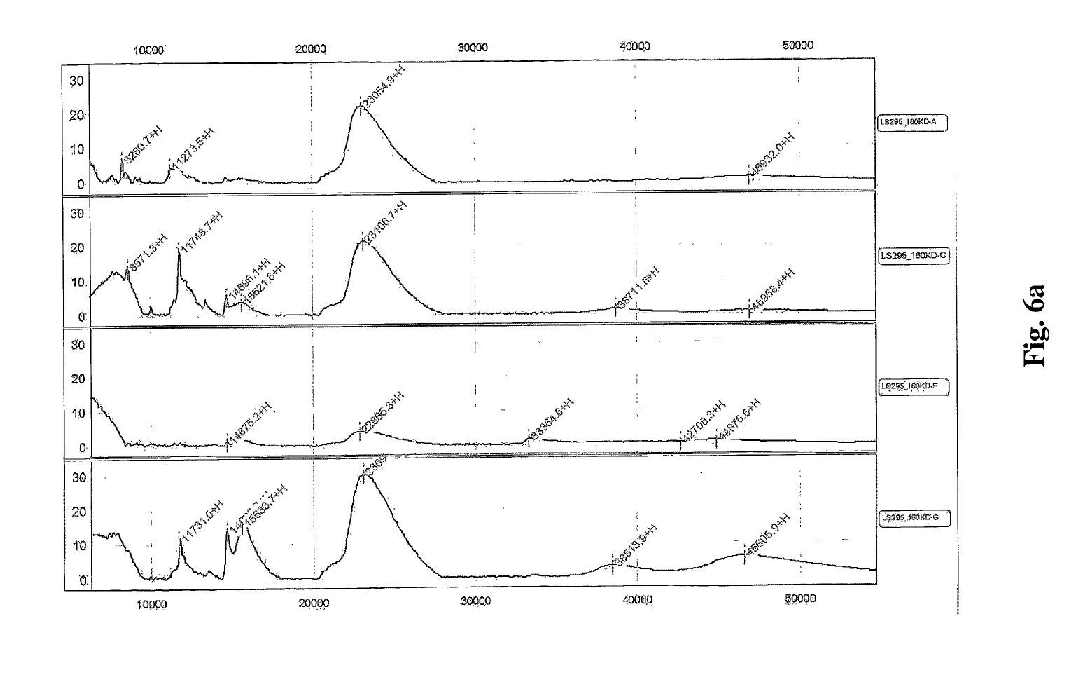

NAF specimens are collected in capillary tubes as above. The capillary tube is broken in half in a 1.7 mL eppendorf tube containing 100 μL Tris buffer, ground with a glass rod, and left overnight at 4° C. The tube is spun down, the supernatant eluted and total protein measured using the Peirce BCA Protein Assay Reagent Kit (Rockford, Ill.). After the total protein concentration for each sample to be analyzed has been determined, the most concentrated samples are diluted with Tris buffer so that each sample has the same total protein concentration. For each specimen, 1-2 μL is pipetted onto the surface of a ProteinChip® (Cyphergen Biosystems, Fremont, Calif.) having probes designed to capture the proteins of interest from NAF. The probes can be either chemical (ionic hydrophobic, hydrophilic, cationic, IMAD, anionic, etc) or biochemical (peptides such as antibodies or receptors, or nucleic acids). After the sample is pipetted onto the chip surface, the surface is washed to remove unbound protein, and an energy absorbing molecule (“EAM”) solution is added on top of each probe. The microarray is then placed in a laser desporption/ionization time-of-flight-mass spectrometer where pulsed nitrogen laser energy, is transmitted through the EAM to ionize proteins from the microarrays and the mass of each protein is measured based on its velocity (time of flight) through the ion chamber. (FIG. 6).

Where two or more proteins of interest have similar chemical properties and mass, the display generally demonstrates broadened peaks or shoulders. A better view of the protein(s) of interest is achieved by using a different chip surface that has greater biding preference for the protein(s) of interest or by washing the spots with a different buffer that preferentially washes away undesired proteins, thus enhancing the signal from the protein of interest.

The subject invention provides a rapid, high throughput means to simply and quickly obtain a screening of transcription, protein expression, and LOH in NAF samples. Only simple hybridization protocols need be employed with the subject microarrays, and signals can be detected using any convenient and readily available detection device. Despite their simplicity, such assays conducted with the subject microarrays yield a large amount of information regarding important breast cancer genes and proteins. As such, the subject microassays find use in a variety of different applications.

What is claimed is:

1. A NAF microarray, comprising a plurality of biopolymeric probes immobilized on a surface of a solid support, wherein each biopolymeric probe corresponds to a gene or fragment of a gene expressed in BECs present in NAF, said gene linked to breast cancer.

2. The microarray of claim 1, wherein said biopolymeric targets are nucleic acids.

3. The microarray of claim 2, wherein said nucleic acids are single stranded polynucleotides.

4. The microarray of claim 2, wherein said biopolymeric targets are microsatellite DNA

5. The microarray of claim 1, wherein the number of biopolymeric targets on said microarray ranges from 50 to 200.

6. A NAF microarray comprising a plurality of 50-200 bioploymeric probes immobilized on a surface of a solid support, wherein each biopolymeric probe comprises a single stranded polynucleotide that corresponds to a gene or fragment of a gene expressed in BECs present in NAF, said gene linked to breast cancer.

7. The microarray of claim 6, wherein a group of apoptosis-related genes are represented on said microarray.

8. The microarray of claim 6, wherein a group of insulin-like growth factor/human kallikrein system genes are represented on said microarray.

9. A NAF microarray comprising a plurality of 50-200 bioploymeric probes immobilized on a surface of a solid support, wherein each biopolymeric probe comprises a single stranded polynucleotide that corresponds to a gene or fragment of a gene expressed in BECs present in NAF, said gene or fragment of a gene linked to breast cancer and further wherein a group of apoptosis-related genes, insulin-like growth factor/human kallikrein system genes, and other key genes of interest or fragments thereof are represented on said microarray.

10. A NAF microarray comprising a plurality of biopolymeric or chemical probes immobilized on a surface of a solid support, wherein each probe binds to a protein present in NAF.

11. The microarray of claim 10, wherein the number of biopolymeric or chemical probes on said microarray ranges from 8 to 200.

12. A NAF microarray comprising a plurality of 8-200 bioploymeric or chemical probes immobilized on a surface of a solid support, wherein each probe binds to a protein present in NAF.

13. The microarray of claim 12, wherein the probes specifically bind to a protein found in NAF.

14. The microarray of claim 13, wherein the probes specifically bind to key NAF proteins.

15. The microaary of claim 14, wherein the probe is a nucleic acid.

16. The microarray of claim 14, wherein the probe is a peptide.

17. A method for detecting alterations in gene expression or gene sequence or copy number in BECs present in NAF, said method comprising contacting a target comprising NAF with an array of biopolymeric probes immobilized on a surface of a solid support; washing said support of unbound target; and detecting a binding event between at least one of said biopolymeric probes and said target.

18. The method of claim 17, wherein said target comprises nucleic acid extracted from BECs isolated from NAF and said binding event comprises hybridization between complementary nucleic acids.

19. The method of claim 17, wherein said target comprises proteins extracted from NAF and said binding event is between peptides of a specific binding pair.

20. The method of claim 17, wherein said target is labeled and said detecting comprises detecting the presence of said label.

21. A kit for use in a method of detecting a binding event between a target comprising NAF and a probe, said kit comprising the NAF microarray of claim 1.

22. The kit of claim 21, wherein said kit further comprises a modified breast pump for collection of NAF.

23. The kit of claim 22, wherein said kit further comprises one or more reagents for use in the assay to be performed.

24. A kit for use in a method of detecting a binding event between a probe comprising NAF and a target, said kit comprising the NAF microarray of claim 10.

25. The kit of claim 21, wherein said kit further comprises a modified breast pump for collection of NAF.

26. The kit of claim 22, wherein said kit further comprises one or more reagents for use in the assay to be performed.

27. A method for individualized screening of the effects of compounds to treat or prevent breast cancer, comprising:
identification of the phenotype of the tumor via microarray analysis of NAF;
selection and administration of a compound to treat or prevent breast cancer; and
analysis of the efficacy of said compound in treating or preventing breast cancer, said analysis comprising collecting NAF and measuring via microarrary analysis differential expression of genes expressed in BECs shed into said NAF or proteins present in said NAF, said gene or protein linked to breast cancer.
